(19) United States
(12) Patent Application Publication
(10) Pub. No.: US 2023/0181820 A1
(43) Pub. Date: Jun. 15, 2023

(54) MICRONEEDLE APPLICATOR

(71) Applicant: JUBILEE BIOTECH CO., LTD., Wonju-si (KR)

(72) Inventors: Sung Kyoung LEE, Seoul (KR); Goonghyun HAN, Siheung-si (KR)

(73) Assignee: JUBILEE BIOTECH CO., LTD., Wonju-si (KR)

(21) Appl. No.: 17/549,355

(22) Filed: Dec. 13, 2021

(51) Int. Cl. A61M 5/142; A61M 37/00; A61M 5/172

(52) U.S. Cl. CPC A61M 5/14248; A61M 37/0015; A61M 5/172; A61M 2037/0023; A61M 2205/8206; A61M 2205/18; A61M 2205/582; A61M 2205/581; A61M 2005/14252

(57) ABSTRACT

A microneedle applicator according to the present disclosure includes a housing which has a cartridge accommodation space formed to detachably mount a cartridge having a microneedle base and which has a first through-hole formed in a bottom surface to allow the microneedle base to pass, a pressing part which is operated to press the microneedle base or seal the first through-hole, a pressing operation part which is configured to operate the pressing part, a cartridge operation part which is configured to rotate the cartridge so that the microneedle base having a microneedle formed at a lower surface thereof is disposed below the pressing part, a controller which is configured to control the pressing operation part and the cartridge operation part and communicate with an external device, and a battery which is configured to supply necessary power for the pressing operation part, the cartridge operation part, and the controller.

### BACKGROUND

### 1. Field of the Invention

The present disclosure relates to a microneedle applicator, and more particularly, to a microneedle applicator capable of infusing a drug periodically or repeatedly while a microneedle cartridge is mounted thereon.

### 2. Discussion of Related Art

Generally, infusing a drug into the skin is referred to as a transdermal drug delivery system. Since the transdermal drug delivery system does not involve the gastrointestinal tract, a drug can be administered regardless of the acidity of the gastrointestinal tract, enzymes in the gastrointestinal tract, food in the gastrointestinal tract, and movement of the gastrointestinal tract.

For the transdermal drug delivery system, ultrasonic waves, jet injection, electroporation, iontophoresis, hypodermic needles, chemical penetration enhancers, microneedles, and the like are used.

Microneedles having a length of hundreds of micrometers deliver a drug component into the skin through the stratum corneum of the skin. A microneedle was developed by Mark Prausnitz in 1998 as a next-generation drug delivery system that combines a conventional syringe with the convenience of a patch to eliminate the fear of needles.

In addition to being able to deliver macromolecular substances such as proteins and peptides, microneedles have advantages such as enabling painless drug delivery, allowing faster recovery of the administration site as compared to general infusions, having a low risk of contamination and infection, and, due to their high effectiveness, allowing the amount of administered drug to be reduced. Accordingly, much research and development for the application of microneedles has taken place.

At an early stage, solid-type microneedles that form fine holes in the skin and allow a drug to penetrate into the skin through the formed holes were widely used. Such solid-type microneedles are still widely used by being processed into the form of a roller in the cosmetic industry.

Microneedles for subcutaneous infusion of drugs are classified into a coated type in which a surface of a needle is coated with a drug, a dissolving type in which a needle itself is formed with a material containing a drug component and the needle is dissolved in the skin, and a hollow type in which a drug is infused through a hollow inside a needle.

U.S. Pat. No. 8,668,675 discloses an applicator that punctures the skin with the hollow-type microneedle to infuse a drug.

The coated-type or dissolving-type microneedle is often manufactured as a patch type, and such a microneedle patch is attached to the skin by hand or by an applicator disclosed in U.S. Pat. No. 10,035,008.

The patch-type and hollow-type microneedles are both for one-time use only and thus have a problem in that periodical or repeated infusion is not possible.

While one infusion is sufficient for medicines such as vaccines, most drugs require repeated administration at predetermined time intervals. An applicator for the hollow-type microneedle lacks such a function, and an applicator for the patch-type microneedles has an inconvenience of requiring an attached patch to be replaced at predetermined time intervals.

Through International Unexamined Patent Application Publication No. WO 2021/167410, the present applicant has disclosed a microneedle applicator and a cartridge that allow a drug to be periodically infused using a microneedle as illustrated in FIGS. 1 to 5.

A microneedle applicator (1) previously filed by the present applicant is a wearable device in the form of a watch. The previously-filed microneedle applicator (1) has an applicator main body (200) having a cartridge (100), which has a plurality of microneedle bases (151) installed therein, mounted thereon and presses one of the microneedle bases (151) toward the skin using a pressing end (404) to puncture the skin with a microneedle (153) disposed at a lower surface of the microneedle base (151) and infuse a drug into the skin.

The cartridge (100) has a plurality of through-holes (150) formed therein, and an inner wall of the through-hole (150) and the microneedle base (151) are connected by a connecting member (155).

The pressing end (404) of the previously-filed microneedle applicator operates by an interaction between a driving source (402) formed of a motor and a power transmitter (403) formed of a gear. Also, the pressing end (404), the driving source (402), and the power transmitter (403) integrally rotate about an operation shaft (450). Due to the rotation, the pressing end (404) moves to above one of the microneedle bases (151), which will infuse a drug, and then moves downward to press the microneedle base (151) toward the skin. Accordingly, the skin is punctured with the microneedle (153) disposed at the lower surface of the microneedle base (151), and the drug is infused into the skin. When drug infusion is completed, the pressing end (404) moves upward, and the microneedle base (151) returns to its original position due to elasticity of the connecting member (155).

In addition to including the driving source (402) and the power transmitter (403) for vertical operation of the pressing end (404) illustrated in FIG. 5, the previously-filed microneedle applicator needs to further include a rotator configured to integrally rotate the pressing end, the driving source, and the power transmitter. Thus, there are problems in that an internal structure of the applicator main body becomes very complex, and the size thereof increases. Also, since a plurality of through-holes should be formed in a housing to allow the microneedle bases to pass, there is a problem in that waterproofing thereof is difficult.

### SUMMARY OF THE INVENTION

The present disclosure is directed to providing a microneedle applicator having a reduced size and a simple structure.

The present disclosure is also directed to providing a microneedle applicator having an excellent waterproof function.

The present disclosure is also directed to providing a microneedle applicator having an excellent antibacterial function.

A microneedle applicator according to the present disclosure includes a housing which has a cartridge accommodation space formed to detachably mount a cartridge having a microneedle base and which has a first through-hole formed in a bottom surface to allow the microneedle base to pass, a pressing part which is moved to press the microneedle base or seal the first through-hole, a pressing operation part which is configured to move the pressing part, a cartridge operation part which is configured to rotate the cartridge so that the microneedle base having a microneedle formed at a lower surface thereof is disposed below the pressing part, a controller which is configured to control the pressing operation part and the cartridge operation part and communicate with an external device, and a battery which is configured to supply necessary power to the pressing operation part, the cartridge operation part, and the controller.

The housing may further include a display part formed on an upper surface.

A contact measurer configured to detect the extent to which the microneedle applicator is in contact with the body of a user may be disposed at a lower surface of the housing.

An antibacterial treatment part may be formed at the lower surface of the housing.

The antibacterial treatment part may have a form that treats the lower surface of the housing with silver or copper or attaches a film coated with silver or copper to the lower surface of the housing.

The first through-hole may be sealed due to the pressing part coming in contact with a second sealing part around the first through-hole.

The housing may be formed of an upper housing and a lower housing detachable from the upper housing, the pressing part, the pressing operation part, the cartridge operation part, and the controller may be disposed in the upper housing, the cartridge accommodation space may be formed between the upper housing and the lower housing, and the first through-hole may be disposed in the lower housing.

The microneedle applicator may further include a first sealing part configured to prevent the penetration of water through a coupling portion between the upper housing and the lower housing.

The upper housing and the lower housing may maintain being coupled by a magnetic force.

The pressing operation part may be formed of a first driving source configured to operate the pressing part and a first power transmitter configured to transmit power from the first driving source.

The first driving source and the first power transmitter may be formed as an electric motor and a gear or may be formed as an electric pump and a cylinder configured to operate using a fluid supplied from the electric pump.

The cartridge operation part may be formed of a second driving source configured to rotate the cartridge and a second power transmitter configured to transmit power from the second driving source.

The pressing operation part and the cartridge operation part may further include an encoder configured to control an amount of rotation.

The microneedle applicator may further include an alarm generator disposed inside the housing to generate an alarm related to operation of the microneedle applicator for the user.

The alarm generator may generate an alarm using one or more of vibration and sound.

In the cartridge, the microneedle base having the microneedle mounted on the lower surface thereof may be provided as one or more microneedle bases.

### BRIEF DESCRIPTION OF THE DRAWINGS

The above and other objects, features and advantages of the present disclosure will become more apparent to those of ordinary skill in the art by describing exemplary embodiments thereof in detail with reference to the accompanying drawings, in which:

FIG. 1 is a view illustrating an exterior of a microneedle applicator according to the related art;

FIG. 2 is a view illustrating the microneedle applicator and a cartridge according to the related art;

FIG. 3 is a view for describing an upper configuration of the cartridge according to the related art;

FIG. 4 is a view for describing a lower configuration of the cartridge according to the related art;

FIG. 5 is a view for describing the operational relationship of the microneedle applicator according to the related art;

FIG. 6 is a perspective view illustrating a microneedle applicator according to an embodiment of the present disclosure;

FIG. 7 is an exploded perspective view of the microneedle applicator according to an embodiment of the present disclosure that is viewed from the top;

FIG. 8 is an exploded perspective view of the microneedle applicator according to an embodiment of the present disclosure that is viewed from the bottom;

FIG. 9 is an exploded perspective view illustrating an internal structure of an upper housing;

FIG. 10 is an exploded perspective view illustrating the internal structure of the upper housing;

FIG. 11A and FIG. 11B are an exploded perspective view illustrating a structure of a pressing operation part;

FIGS. 12 and 13 are views illustrating examples of a cartridge according to an embodiment of the present disclosure;

FIGS. 14A, 14B, 15A, 15B, 15C, 16A, 16B, 17A and 17B are views for describing the operational relationship of a pressing part according to an embodiment of the present disclosure; and

FIG. 18 is a view illustrating a cartridge operation part according to an embodiment of the present disclosure.

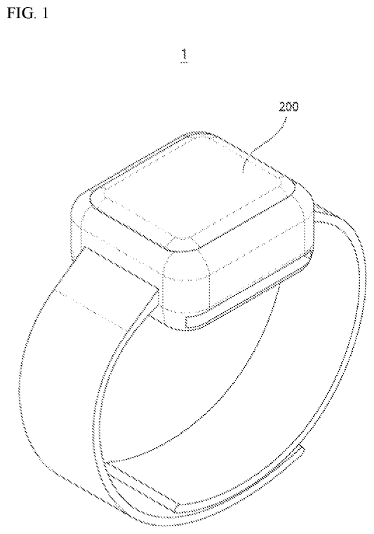

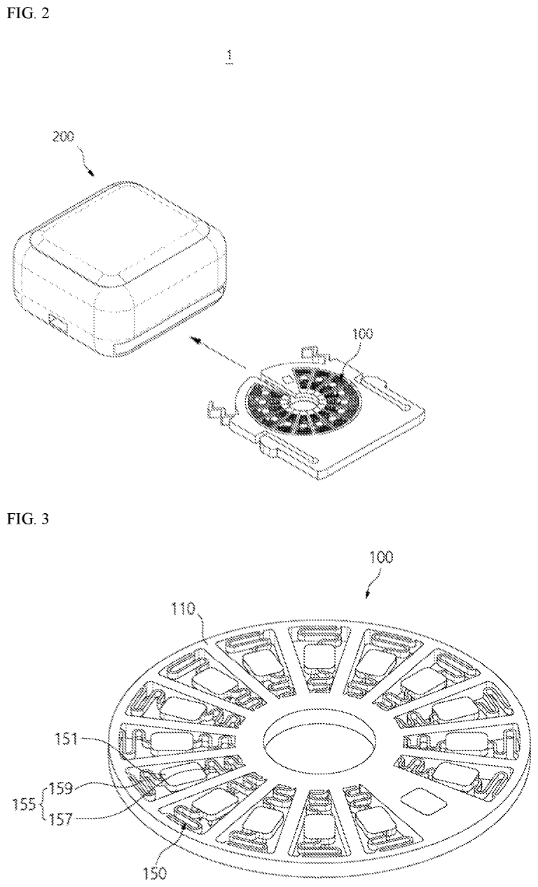

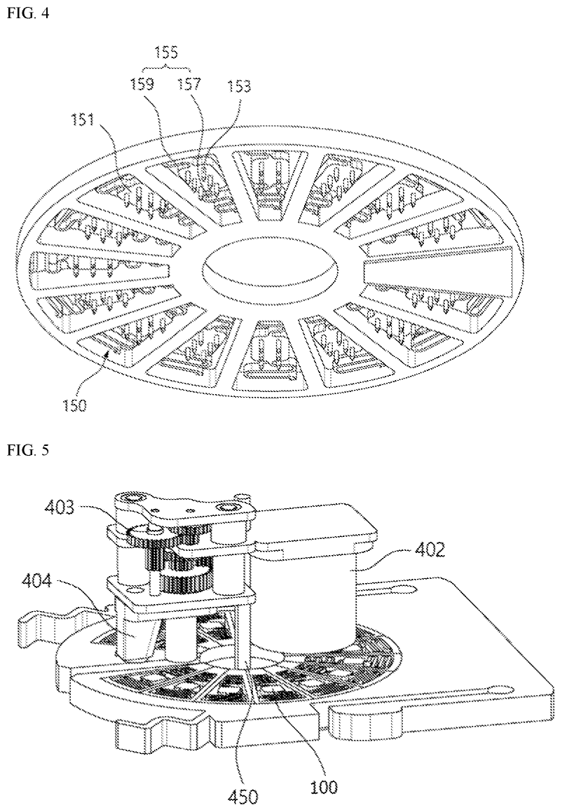

### DETAILED DESCRIPTION OF EXEMPLARY EMBODIMENTS

Hereinafter, embodiments of the present disclosure will be described in detail with reference to the accompanying drawings.

A microneedle applicator according to the present disclosure is a wearable device and may be in the form of a wristwatch that is worn around the wrist or may be in the form of a band that is worn around a part of the body such as a forearm, a thigh, or the waist.

The microneedle applicator according to the present disclosure is not limited to being used for infusing a drug into human skin and may also be used for infusing a drug into animal skin. For example, the microneedle applicator may also be manufactured as a wearable device that is in the form of a strap worn around the neck of an animal.

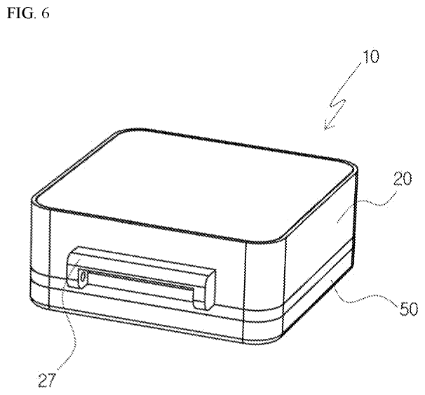

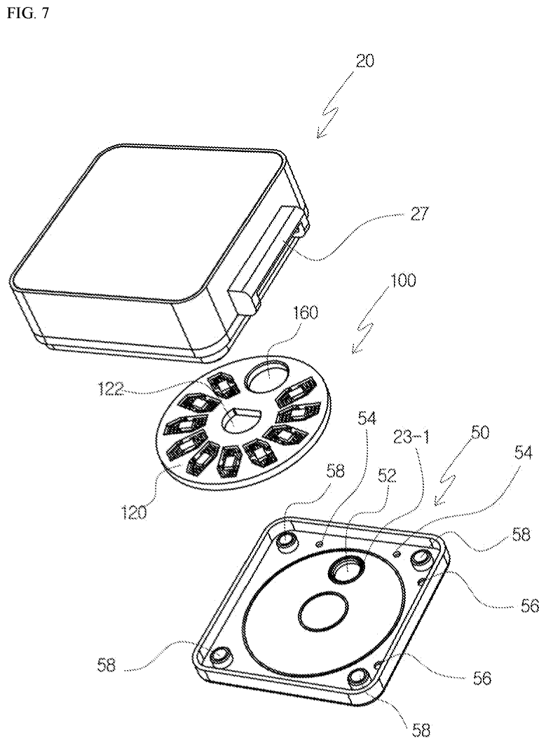

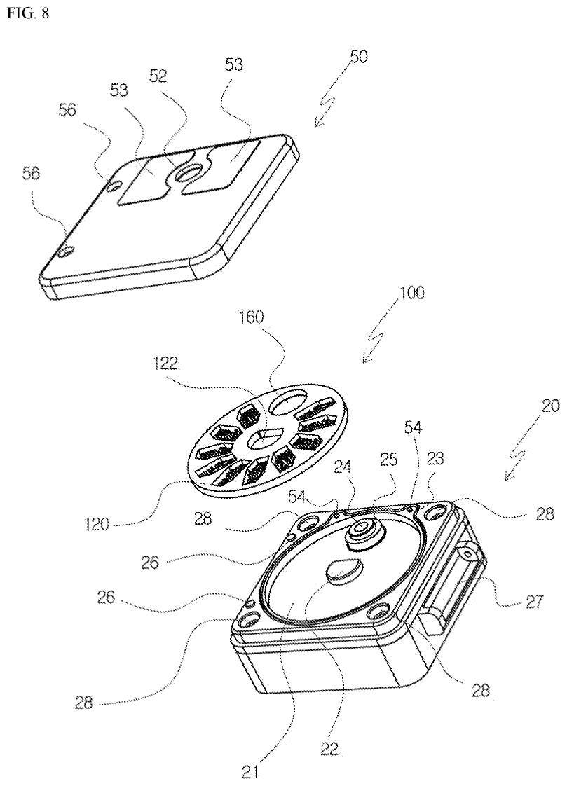

FIG. 6 illustrates an applicator main body according to an embodiment of the present disclosure, and a strap mounting part 27 is disposed at both sides of the applicator main body to connect a strap which will be used when wearing the microneedle applicator around the wrist.

The microneedle applicator according to the present embodiment is divided into an upper housing 20 and a lower housing 50.

In the upper housing 20, a pressing part, a pressing operation part 500 configured to operate the pressing part, a cartridge operation part 600 configured to rotate a cartridge 100, and a controller 700 configured to control each of the operation parts and communicate with an external device are disposed.

A display part configured to notify a user of operational information, an operation time, etc. of the microneedle applicator may be disposed on an upper surface of the upper housing 20.

Also, the display part may be provided as a touchscreen to allow the user to manipulate the microneedle applicator by touching a surface of the display part.

A cartridge accommodation space 21 configured to store the cartridge 100 is formed between the lower housing 50 and the upper housing 20.

A charging terminal 26 is disposed on a lower surface of the upper housing 20, and a battery 800 may be charged through the charging terminal 26. Also, a first sealing part 23 is disposed along an outer boundary of the cartridge accommodation space 21, and the first sealing part 23 prevents the penetration of moisture through a boundary surface between the upper housing 20 and the lower housing 50.

The upper housing 20 and the lower housing 50 respectively include coupling parts 28 and 58 which are coupled to each other by a magnetic force. In this way, the upper housing 20 and the lower housing 50 are coupled to each other.

The upper housing 20 and the lower housing 50 may also be coupled to each other by any other known coupling means.

In the lower housing 50, a first through-hole 52 to which first and second pressing parts 24 and 25 are coupled and a charging terminal hole 56 configured to expose the charging terminal 26 to the outside are formed.

Also, a contact measurer 53 is disposed on a lower surface of the lower housing 50. The contact measurer 53 is formed of an electrostatic element or a piezoelectric element and enables measuring whether the microneedle applicator is in close contact with the wearer's skin. The contact measurer 53 is controlled by the controller 700 disposed in the upper housing 20 and exchanges a control signal through a terminal 54.

Also, an antibacterial treatment part may be formed on the lower surface of the lower housing 50, and the antibacterial treatment part may be manufactured to perform surface treatment by coating a lower surface of a housing with silver or copper or causing a material of the housing to include silver or copper. Alternatively, the antibacterial treatment part may be formed to attach a film coated with silver or copper to a lower surface of a housing. Accordingly, the antibacterial treatment part may prevent an occurrence of contamination between the microneedle applicator and the skin when the microneedle applicator is worn in close contact with the skin.

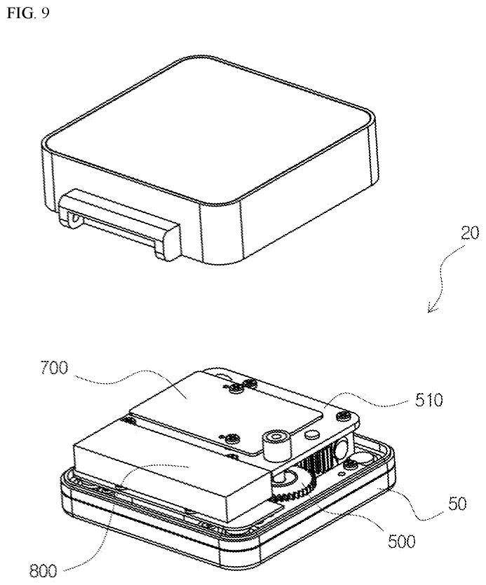

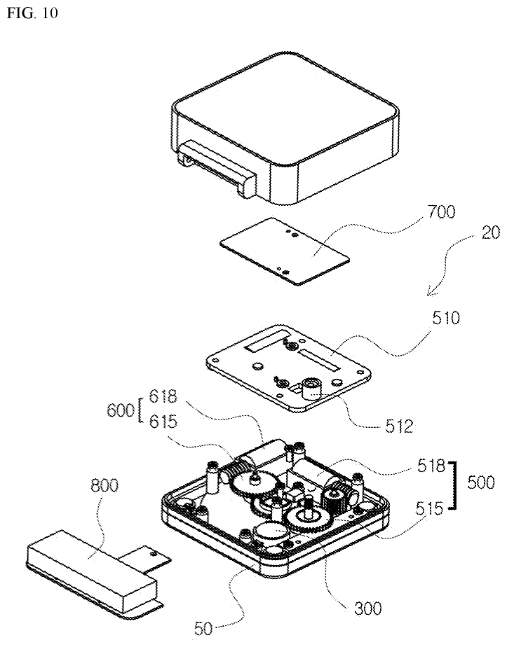

As illustrated in FIGS. 9 and 10, the pressing operation part 500, the cartridge operation part 600, the controller 700 on which electronic parts necessary for a printed circuit board are mounted, and the battery 800 are disposed in the upper housing 20.

Also, an alarm generator 300 configured to notify the user of an operation of the microneedle applicator is disposed in the upper housing 20. The alarm generator 300 may generate an alarm using one or more of vibration and sound.

The alarm generator 300 may also be used for the purpose of generating vibrations to promote the dissolution of a drug of the microneedle after the skin is punctured with the microneedle.

FIG. 11 is an exploded perspective view illustrating a structure of the pressing operation part 500, and FIGS. 14 to 17 are views only illustrating parts necessary to describe operation of the first and second pressing parts 24 and 25 due to the pressing operation part 500.

A first step 24-1 is formed on one end of the first pressing part 24, and a first male screw 514 is processed on the other side thereof. Also, a first gear 515 is installed at the center of the first pressing part 24 and rotates along with the first pressing part 24.

A screw support hole 512 is formed in an intermediate plate 510 fixed inside the upper housing, and a first female screw 513 is fixed inside the screw support hole 512.

The first male screw 514 of the first pressing part 24 is coupled to the first female screw 513. When the first pressing part 24 rotates, the first pressing part 24 vertically moves due to the first female screw 513 fixed to the first male screw 514 which rotates.

In the pressing operation part 500, when a first motor 518 rotates, a worm-type first motor gear 517 which is coupled to a shaft of the first motor 518 rotates, and a worm-type second gear 516 coupled to the first motor gear 517 rotates.

The first gear 515 is coupled to the second gear 516, and the first gear 515 moves upward and downward along with the first pressing part 24 while rotating. In order to maintain being coupled to the first gear 515 even while the first gear 515 moves upward and downward, the second gear 516 has a height equivalent to a range in which the first gear 515 moves upward and downward.

The first step 24-1 formed on the one end of the first pressing part 24 is engaged with a second step 25-1 of the second pressing part 25.

The second pressing part 25 is always pressed toward the first step 24-1 due to a spring 520 disposed below the first gear 515.

The first pressing part 24 and the second pressing part 25 will be collectively referred to as “pressing part” as necessary.

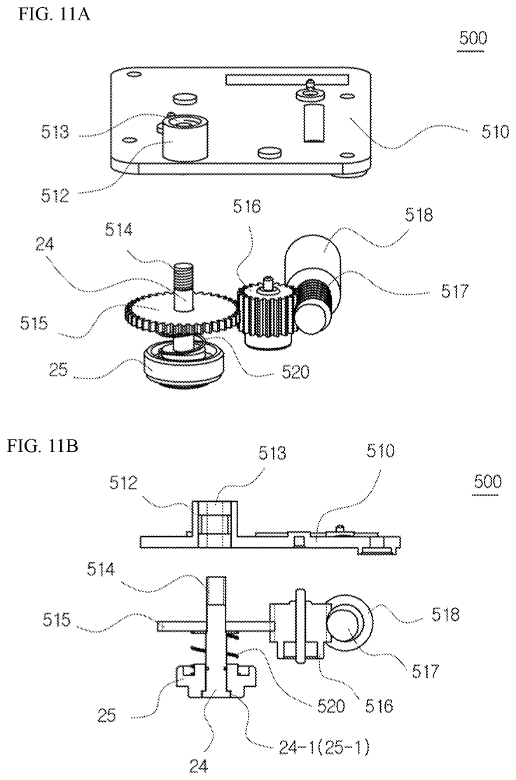

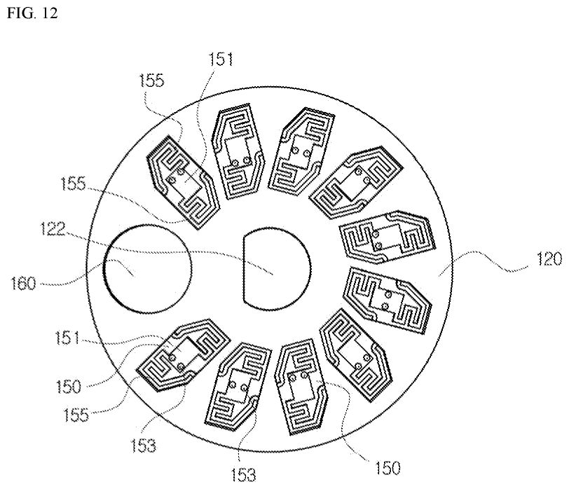

As illustrated in FIG. 12, in the cartridge mounted on the microneedle applicator according to an embodiment of the present disclosure, a plurality of through-holes 150 are formed in a cartridge main body 120. A microneedle base 151 having the microneedle 153 disposed at a lower surface thereof is disposed in each through-hole 150 by a connecting member 155. This is the same as in the cartridge of the related art filed by the present applicant.

The microneedle applicator according to the present disclosure rotates the cartridge inserted thereinto, and the pressing part pressing the microneedle base 151 seals the first through-hole 52. Thus, the cartridge according to an embodiment that is illustrated in FIG. 12 further includes a rotational force transmitter 122, in which a through-hole to be coupled to a coupling shaft 22 is formed, and a pressing part passing hole 160 through which the first and second pressing parts 24 and 25 pass.

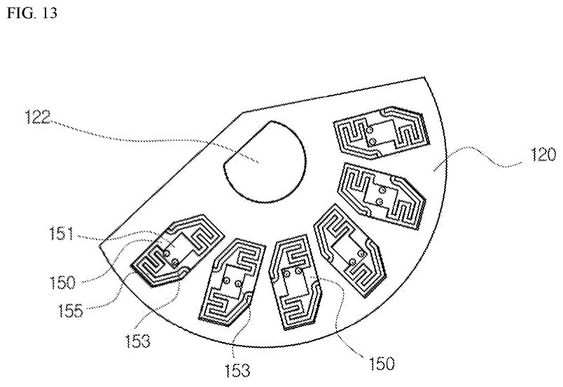

The rotational force transmitter 122 may be, instead of being configured to be coupled to the coupling shaft at the center of the cartridge, formed as a gear along the circumference of the cartridge main body 120, and instead of the cartridge including the pressing part passing hole 160, the shape of the cartridge main body 120 may be changed to the shape illustrated in FIG. 13 so that interference does not occur when the pressing part seals the first through-hole.

Also, although the cartridges illustrated in FIGS. 12 and 13 include a plurality of microneedle bases, a cartridge including only one microneedle base may also be applied to the microneedle applicator according to the present disclosure.

That is, a cartridge applied to the microneedle applicator according to the present disclosure may have any form as long as the cartridge is able to puncture the skin with a microneedle through the first through-hole, and a pressing part configured to press the microneedle is able to seal the first through-hole.

FIG. 14 illustrates the operational relationship in which the first and second pressing parts 24 and 25 move downward and seal the first through-hole 52 of the lower housing 50.

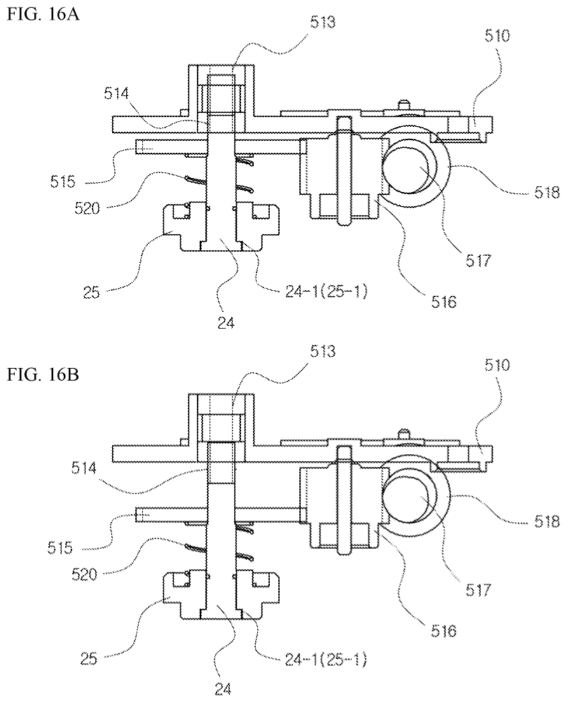

FIG. 14A illustrates a state in which the pressing part passing hole 160 of the cartridge main body 120 is disposed at lower ends of the first and second pressing parts 24 and 25. In this state, as illustrated in FIG. 16A, the first gear 515 is at the most elevated position, and the second pressing part 25 is pressed toward the first step 24-1 of the first pressing part 24 by the spring 520.

FIG. 14B illustrates a state in which the first and second pressing parts 24 and 25 block the first through-hole 52 of the lower housing 50 due to moving downward while passing through the pressing part passing hole 160. In this state, as illustrated in FIG. 16B, the first and second pressing parts 24 and 25 move downward to a position where the first and second pressing parts 24 and 25 seal the first through-hole.

Sealing of the first through-hole 52 is performed by the second pressing part 25 pressing a second sealing part 23-1 around the first through-hole 52, and sealing between the first pressing part 24 and the second pressing part 25 is performed by a third sealing part 23-2.

As described above, the first to third sealing parts 23, 23-1, and 23-2 may prevent the penetration of water into the upper housing 20.

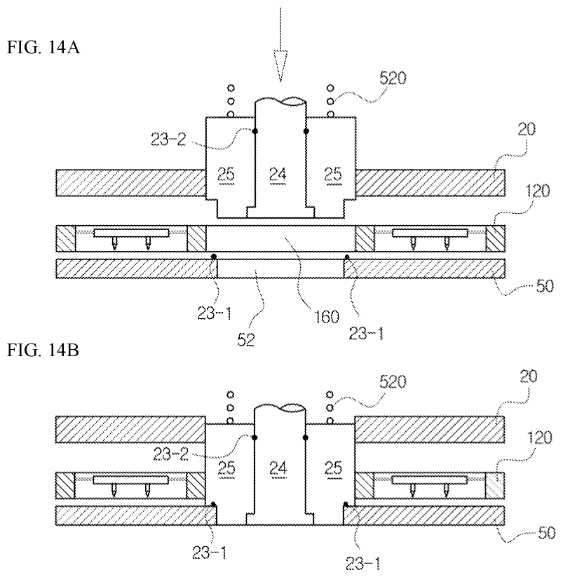

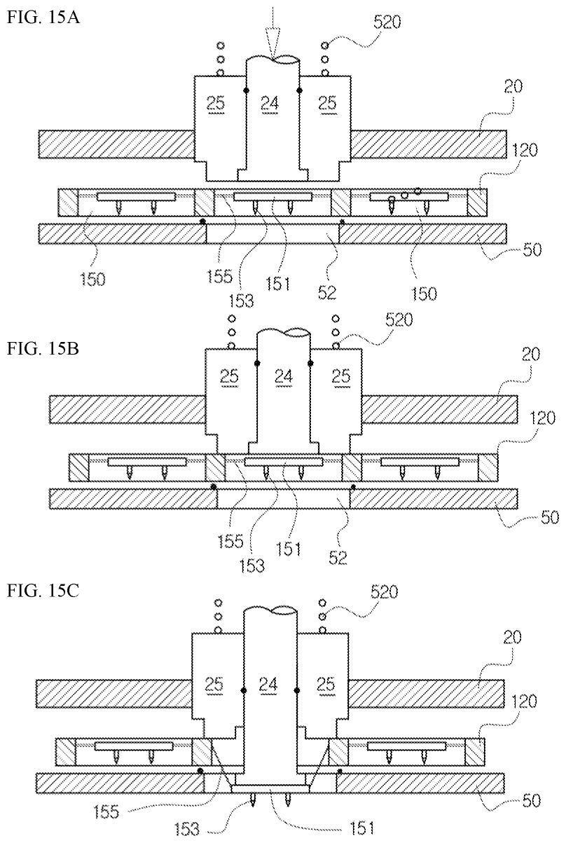

FIG. 15A illustrates a state in which the microneedle base 151 of the cartridge main body 120 is disposed at the lower ends of the first and second pressing parts 24 and 25. In this state, as illustrated in FIG. 16A, the first gear 515 is at the most elevated position, and the second pressing part 25 is pressed toward the first step 24-1 of the first pressing part 24 by the spring 520.

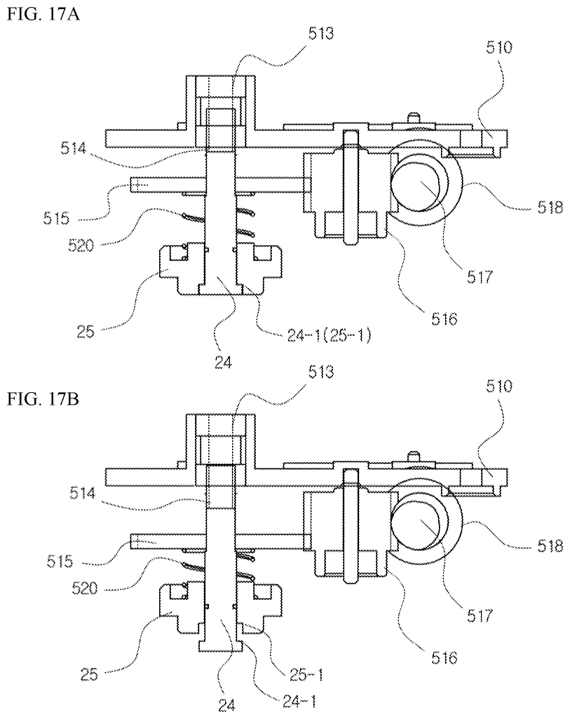

When the first and second pressing parts 24 and 25 move downward as in FIG. 17A, a lower surface of the second pressing part 25 comes in contact with the cartridge main body 120 as illustrated in FIG. 15B.

Then, movement of the second pressing part 25 is restricted by the cartridge main body 120, and only the first pressing part 24 moves further downward as in FIG. 17B and presses the microneedle base 151. Accordingly, as in FIG. 15C, it becomes possible to puncture the skin with the microneedle 153 and infuse a drug into the skin.

Although the first pressing part 24 moves upward and downward while rotating, various conditions that allow friction of an interface with the microneedle base to be reduced, that is, application of a lubricant, a film on an upper surface of the cartridge, a structure of the connecting member of the microneedle base, installation of a rotation preventing member at a distal end of the first pressing part, all of which may reduce the coefficient of friction, may allow the microneedle base to move in the vertical direction.

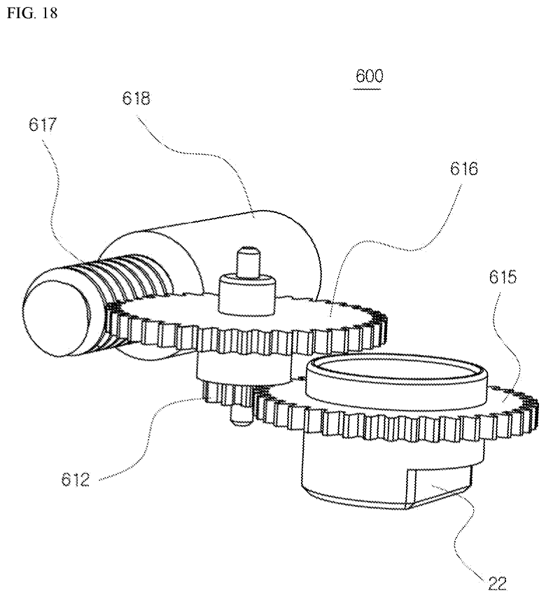

As illustrated in FIG. 18, in the cartridge operation part 600 according to the present embodiment, a rotational force of a second motor 618 is transmitted to a fourth gear 616, which is a worm gear, through a second motor gear 617, which is a worm gear. A rotational force of the fourth gear 616 is reduced by a fifth gear 612 installed at the same shaft and then is transmitted to a third gear 615.

Since the third gear 615 is coaxial with the coupling shaft 22 which will be coupled to the rotational force transmitter 122 of the cartridge, the cartridge may rotate due to the rotational force of the second motor 618.

The rotation of the pressing operation part 500 and the cartridge operation part 600 may be controlled by installing an encoder on one of the motors or gears thereof.

In the above description, the pressing operation part 500 includes the first driving source formed of the first motor 518 and the first power transmitter formed of the first and second gears 515 and 516. However, the pressing operation part may also be configured such that the first driving source is an electric pump and the first power transmitter is a cylinder that operates using a fluid supplied from the electric pump.

Also, for the cartridge operation part, various other known driving sources and power transmitters may be adopted in configuring the second driving source configured to rotate the cartridge and the second power transmitter configured to transmit power from the second driving source.

Also, the microneedle applicator according to the present disclosure is more suitable for the dissolving-type or coated-type microneedle.

The present disclosure can provide a microneedle applicator that has a reduced size and a simple structure and has excellent waterproof and antibacterial functions.

The above description of the embodiments has been provided for the purpose of describing the principles of the present disclosure and actual applications thereof, and from the above description, those of ordinary skill in the art should understand that the present disclosure may include various embodiments and various modifications.

The disclosed embodiments are not intended to limit the present disclosure, and any of the embodiments and/or elements disclosed herein may be combined with each other to form various other embodiments not specifically disclosed herein. Therefore, additional embodiments are possible, and such embodiments also belong to the scope of the present disclosure.

What is claimed is:

1. A microneedle applicator comprising:
a housing which has a cartridge accommodation space formed to detachably mount a cartridge having a microneedle base and which has a first through-hole formed in a bottom surface to allow the microneedle base to pass;
a pressing part which is moved to press the microneedle base or seal the first through-hole;
a pressing operation part which is configured to move the pressing part;
a cartridge operation part which is configured to rotate the cartridge so that the microneedle base having a microneedle formed at a lower surface thereof is disposed below the pressing part;
a controller which is configured to control the pressing operation part and the cartridge operation part and communicate with an external device; and
a battery which is configured to supply necessary power to the pressing operation part, the cartridge operation part, and the controller.

2. The microneedle applicator of claim 1, wherein the housing further includes a display part formed on an upper surface.

3. The microneedle applicator of claim 1, wherein a contact measurer configured to detect the extent to which the microneedle applicator is in contact with the body of a user is disposed at a lower surface of the housing.

4. The microneedle applicator of claim 1, wherein an antibacterial treatment part is formed at a lower surface of the housing.

5. The microneedle applicator of claim 4, wherein the antibacterial treatment part has a form that treats the lower surface of the housing with silver or copper or attaches a film coated with silver or copper to the lower surface of the housing.

6. The microneedle applicator of claim 1, wherein the first through-hole is sealed due to the pressing part coming in contact with a second sealing part around the first through-hole.

7. The microneedle applicator of claim 1, wherein the housing is formed of an upper housing and a lower housing detachable from the upper housing, the pressing part, the pressing operation part, the cartridge operation part, and the controller are disposed in the upper housing, the cartridge accommodation space is formed between the upper housing and the lower housing, and the first through-hole is disposed in the lower housing.

8. The microneedle applicator of claim 7, further comprising a first sealing part configured to prevent the penetration of water through a coupling portion between the upper housing and the lower housing.

9. The microneedle applicator of claim 7, wherein the upper housing and the lower housing maintain being coupled by a magnetic force.

10. The microneedle applicator of claim 1, wherein the pressing operation part is formed of a first driving source configured to operate the pressing part and a first power transmitter configured to transmit power from the first driving source.

11. The microneedle applicator of claim 10, wherein the first driving source and the first power transmitter are formed as an electric motor and a gear or are formed of an electric pump and a cylinder configured to operate using a fluid supplied from the electric pump.

12. The microneedle applicator of claim 1, wherein the cartridge operation part is formed of a second driving source configured to rotate the cartridge and a second power transmitter configured to transmit power from the second driving source.

13. The microneedle applicator of claim 1, wherein the pressing operation part and the cartridge operation part further include an encoder configured to control an amount of rotation.

14. The microneedle applicator of claim 1, further comprising an alarm generator disposed inside the housing to generate an alarm related to operation of the microneedle applicator for the user.

15. The microneedle applicator of claim 14, wherein the alarm generator generates an alarm using one or more of vibration and sound.

16. The microneedle applicator of claim 1, wherein, in the cartridge, the microneedle base having the microneedle mounted on the lower surface thereof is provided as one or more microneedle bases.
